(19) United States
(12) Patent Application Publication
(10) Pub. No.: US 2026/0170873 A1
(43) Pub. Date: Jun. 18, 2026

(54) DETERMINATION METHOD, DETERMINATION DEVICE, AND DETERMINATION SYSTEM

(71) Applicant: Panasonic Intellectual Property Management Co., Ltd., Osaka (JP)

(72) Inventors: Kengo WADA, Osaka (JP); Takahiro AIHARA, Osaka (JP); Yoshihiro MATSUMURA, Osaka (JP); Fumihiro NARUSE, Kyoto (JP); Chie MINAMI, Hyogo (JP); Ryota SUZUKI, Ehime (JP)

(73) Assignee: Panasonic Intellectual Property Management Co., Ltd., Osaka (JP)

(21) Appl. No.: 18/711,804

(22) PCT Filed: Nov. 17, 2022
(86) PCT No.: PCT/JP2022/042776

§ 371 Date: May 20, 2024

(30) Foreign Application Priority Data
Dec. 3, 2021 (JP) 2021-197216

(51) Int. Cl. G06V 40/20

(52) U.S. Cl. CPC G06V 40/28

(57) ABSTRACT

A determination method according to the present embodiment includes: estimating, based on an image that includes, as a subject, a target person performing a specific action, a skeletal model of the target person in the image; setting a plurality of three-dimensional regions around the skeletal model based on positions of a plurality of skeletal points in the skeletal model; identifying, among the plurality of three-dimensional regions, a three-dimensional region where a skeletal point of a wrist included in the plurality of skeletal points is located in the specific action; and determining, based on the three-dimensional region identified in the identifying, a degree to which the target person is capable of performing an activity of daily living.

### TECHNICAL FIELD

The present invention relates to determination methods, determination devices, and determination systems.

### BACKGROUND ART

Conventionally, nursing homes provide training (so-called rehabilitation) services so that elderly people can live independently. A staff member at the nursing home who is qualified to produce a training plan visits the home of an elderly person to determine the physical function and the state of activities of daily living (ADL) of the elderly person and to produce a training plan corresponding to the state of the ADL. The rehabilitation is performed according to the training plan which has been produced.

For example, Patent Literature (PTL) 1 discloses an action information processing device which acquires, in the evaluation of rehabilitation, action information of a target person who performs a predetermined action, analyzes the acquired action information, and displays display information based on an analysis value related to the movement of a specified part.

### CITATION LIST

### Patent Literature

[PTL 1] Japanese Unexamined Patent Application Publication No. 2015-061579

### SUMMARY OF INVENTION

### Technical Problem

In order to produce a training plan of effective rehabilitation for a target person, it is necessary to accurately determine the state of activities of daily living of the target person. It is desirable to be able to easily determine the state of activities of daily living of the target person.

The present invention provides a determination method, a determination device, and a determination system which can easily and accurately determine the state of activities of daily living of a target person.

### Solution to Problem

A determination method according to an aspect of the present invention is a determination method performed by a computer, and includes: estimating, based on an image that includes a target person as a subject, a skeletal model of the target person in the image, target person performing a specific action; setting a plurality of three-dimensional regions around the skeletal model based on positions of a plurality of skeletal points in the skeletal model; identifying, among the plurality of three-dimensional regions, a three-dimensional region where a skeletal point of a wrist included in the plurality of skeletal points is located in the specific action; and determining, based on the three-dimensional region identified in the identifying, a degree to which the target person is capable of performing an activity of daily living.

A determination device according to an aspect of the present invention includes: an estimator that estimates, based on an image which includes, as a subject, a target person performing a specific action, a skeletal model of the target person in the image; a setter that sets a plurality of three-dimensional regions around the skeletal model based on positions of a plurality of skeletal points in the skeletal model; an identifier that identifies, among the plurality of three-dimensional regions, a three-dimensional region where a skeletal point of a wrist included in the plurality of skeletal points is located in the specific action; and a determiner that determines, based on the three-dimensional region identified by the identifier, a degree to which the target person is capable of performing an activity of daily living.

A determination system according to an aspect of the present invention includes: the determination device described above; and an information terminal, the determination device further includes: a first communicator that communicates with the information terminal; an acquirer that acquires the image from the information terminal via the first communicator; and an outputter that outputs a result of determination made by the determiner to the information terminal via the first communicator, and the information terminal includes a second communicator that communicates with the determination device, an instructor that instructs the target person to perform the specific action, a camera that generates the image by capturing an image of the target person performing the specific action, a controller that outputs the image generated to the determination device via the second communicator, and acquires the result of the determination from the determination device via the second communicator, and a presenter that presents the result of the determination.

### Advantageous Effects of Invention

According to the present invention, a determination method, a determination device, and a determination system which can easily and accurately determine the state of activities of daily living of a target person are realized.

### BRIEF DESCRIPTION OF DRAWINGS

FIG. 1 is a block diagram showing the functional configuration of a determination system according to an embodiment.

FIG. 2 is a diagram for illustrating a skeletal model of a target person which is estimated by an estimator in the embodiment.

FIG. 3 is a diagram for illustrating three-dimensional regions which are set by a setter in the embodiment.

FIG. 4 is a diagram for illustrating a three-dimensional region which is identified by an identifier in the embodiment.

FIG. 5 is a diagram showing specific examples of criteria which are used by a determiner in the embodiment.

FIG. 6 is a diagram showing a specific example of a result of a determination which is presented by a presenter in the embodiment and is made by the determiner.

FIG. 7 is a flowchart showing a processing procedure which is performed by a determination device according to the embodiment.

FIG. 8 is a flowchart showing a processing procedure which is performed by a determination system according to the embodiment.

### DESCRIPTION OF EMBODIMENTS

Embodiments will be specifically described below with reference to drawings. Each of the embodiments described below indicates a comprehensive or specific example. Numerical values, shapes, materials, constituent elements, the arrangement and connection of the constituent elements, steps, the order of the steps, and the like shown in the following embodiments are examples, and are not intended to limit the present invention. Among the constituent elements in the following embodiments, constituent elements which are not recited in the independent claims are described as optional constituent elements.

The drawings are schematic views and are not exactly shown. In the drawings, substantially the same configurations are identified with the same reference signs, and repeated description may be omitted or simplified.

### Embodiment

### [Configuration]

The configuration of a determination system according to an embodiment will first be described.

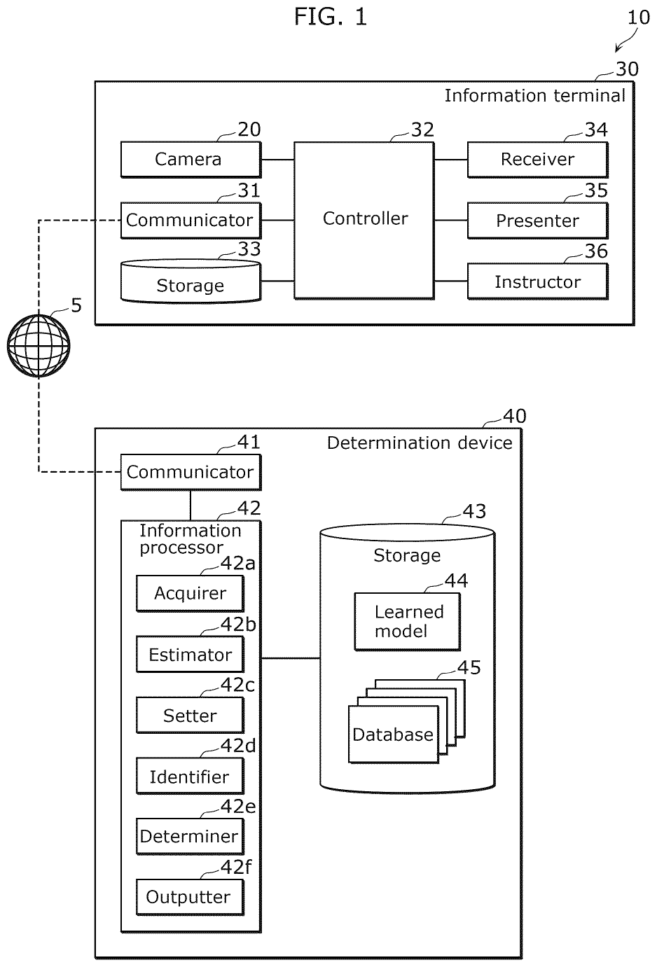

FIG. 1 is a block diagram showing the functional configuration of determination system 10 according to the embodiment.

Determination system 10 is a system which determines, based on an image which includes, as a subject, a target person performing a specific action (that is, an image in which the target person is shown), the degree to which the target person is capable of performing an activity of daily living (ADL).

Determination system 10 includes information terminal 30 and determination device 40.

For example, a user operates information terminal 30 to capture an image of the target person. The image (more specifically, moving images) generated in this way is transmitted to determination device 40. Determination device 40 determines, based on the received image, the degree to which the target person who is included in the image as the subject is capable of performing an activity of daily living. The result of the determination is transmitted to information terminal 30, and is presented by information terminal 30 to the user. For example, determination device 40 evaluates, in a multilevel manner, the degree to which the target person is capable of performing an activity of daily living using a five-level evaluation from 1 to 5, a numerical value such as 0%, 50%, 75%, or 100%, or a symbol such as A, B, or C.

Here, the target person is a person with the degree of an activity of daily living capable of being performed being determined, and is, for example, a person whose physical function, that is, the ability to move the body, is impaired due to illness, trauma, aging, or disability.

Examples of the user include a physical therapist, an occupational therapist, a nurse, and a rehabilitation specialist.

Activities of daily living are the minimum daily activities necessary for daily life. Activities of daily living include, for example, getting up, transferring, moving, eating, putting on and taking off shoes, changing clothes such as putting on clothes, toileting, bathing such as washing hair, and grooming.

Specific actions are actions associated with activities of daily living. For example, specific actions are actions which are common or similar to at least part of actions included in activities of daily living. Specific examples of the specific action will be described later.

Information terminal 30 is a computer which instructs the target person to perform the specific action, acquires an image (image data) that is generated by capturing an image of the target person with camera 20 and includes the target person as the subject, and transmits the acquired image to determination device 40. In the present embodiment, information terminal 30 captures images of the target person to generate moving images including a plurality of images, and transmits the generated moving images to determination device 40.

Information terminal 30 is, for example, a portable computer device, such as a smartphone or a tablet terminal, which is used by the user. Information terminal 30 may be a stationary computer device such as a personal computer.

Information terminal 30 includes camera 20, communicator 31, controller 32, storage 33, receiver 34, presenter 35, and instructor 36.

Camera 20 is a camera which captures an image of the target person who performs the specific action to generate an image that includes, as the subject, the target person performing the specific action. In the present embodiment, camera 20 is a video camera which captures images of the target person performing the specific action to generate moving images (that is, moving images that include a plurality of images each including the target person as the subject) that include, as the subject, the target person performing the specific action. Camera 20 may be a camera which uses a complementary metal oxide semiconductor (CMOS) image sensor or may be a camera which uses a charge coupled device (CCD) image sensor.

Camera 20 may be an external camera which is attached to information terminal 30. In this case, information terminal 30 does not need to include camera 20, and may include a communication interface for connecting to camera 20 to be able to communicate with camera 20.

Communicator 31 is a communication interface which communicates with determination device 40. Specifically, information terminal 30 uses communicator 31 to communicate with determination device 40 via network 5 such as the Internet. Communicator 31 is an example of a second communicator. Communicator 31 is realized, for example, by a wireless communication circuit for performing wireless communication with determination device 40.

The communication standard of communication performed by communicator 31 is not particularly limited.

Communicator 31 may be connected to determination device 40 to be able to perform wireless communication with determination device 40 or may be connected to determination device 40 to be able to perform wired communication with determination device 40. For example, when communicator 31 is connected to determination device 40 to be able to perform wired communication with determination device 40, the wired communication is realized, for example, by a connector connected to a communication line or the like.

Controller 32 is a processor which performs various types of information processing in information terminal 30. For example, controller 32 outputs an image generated by camera 20 to determination device 40 via communicator 31. For example, when controller 32 outputs moving images, controller 32 outputs images together with a plurality of images for calibrating the moving images such that each of the images is associated with information of a time at which the image is generated. For example, controller 32 acquires, from determination device 40, via communicator 31, the result of a determination (determination result information) made by determination device 40 (more specifically, determiner 42e). For example, controller 32 causes presenter 35 to present information indicating the acquired result of the determination. Controller 32 performs various types of processing, for example, based on operation inputs received by receiver 34. For example, controller 32 is realized by a microcomputer. Controller 32 may also be realized by a processor. For example, the microcomputer, the processor, or the like of controller 32 executes dedicated application programs stored in storage 33 to realize the functions of controller 32.

Storage 33 is a storage device in which the dedicated application programs and the like to be executed by controller 32 are stored. Storage 33 is realized, for example, by a semiconductor memory, a hard disk drive (HDD), or the like.

Receiver 34 is an input interface which receives an operation input performed by the user of information terminal 30 (for example, a rehabilitation specialist). For example, receiver 34 receives an input operation performed by the user for providing, for example, an instruction to start processing for determining the degree to which the target person is capable of performing an activity of daily living. Receiver 34 is realized, for example, by a touch panel display or the like. For example, when receiver 34 is realized by a touch panel display, the touch panel display functions as presenter 35 and receiver 34.

Receiver 34 is not limited to a touch panel display, and may be, for example, a keyboard, a pointing device such as a touch pen or a mouse, a hardware button, or the like. When receiver 34 receives an input of a voice, receiver 34 may be a microphone. When receiver 34 receives an input of a gesture, receiver 34 may be a camera. In this case, receiver 34 may be realized by camera 20 or may be realized by a camera different from camera 20.

Presenter 35 is a presentation device which presents the result of the determination made by determination device 40. Specifically, presenter 35 presents information indicating the degree of the state of activities of daily living of the target person which is determined by determination device 40.

The form of the presentation of the information to the user which is performed by presenter 35 is not particularly limited. Presenter 35 may present the information to the user using a picture (an image or moving images), may present the information to the user using a voice, or may present the information to the user using a picture and a voice. For example, presenter 35 may be realized by a display panel such as a liquid crystal panel or an organic electro luminescence (EL) panel, may be realized by a sound device such as a loudspeaker or an earphone, or may be realized by a display panel and a sound device.

Instructor 36 is an instruction device which instructs the target person to perform the specific action. Instructor 36 uses, for example, a picture, a voice, or the like to instruct the target person to perform the specific action. In other words, instructor 36 may use a picture to provide an instruction to the user or may use a voice to provide an instruction to the user.

For example, instructor 36 uses a picture and/or a voice to provide an instruction such as “take a banzai action”, “touch your back and maintain that posture”, “touch the back of your head and maintain that posture”, or “touch toes and maintain that posture”. For example, instructor 36 may be realized by a display panel such as a liquid crystal panel or an organic EL panel, may be realized by a sound device such as a loudspeaker or an earphone, or may be realized by a display panel and a sound device.

Instructor 36 and presenter 35 may be realized by the same display panel and/or the same device such as a sound device.

Picture information and/or voice information used by instructor 36 for instructing the target person to perform the specific action may be previously stored in storage 33.

Determination device 40 is a computer which acquires the image transmitted from information terminal 30, estimates a skeletal model of the target person in the acquired image, and determines the state of activities of daily living of the target person based on the estimated skeletal model.

Determination device 40 includes communicator 41, information processor 42, and storage 43.

Communicator 41 is a communication interface which communicates with information terminal 30. Specifically, determination device 40 uses communicator 41 to communicate with information terminal 30 via network 5 such as the Internet. Communicator 41 is an example of a first communicator. Communicator 41 Is realized, for example, by a wireless communication circuit for performing wireless communication with information terminal 30.

The communication standard of communication performed by communicator 41 is not particularly limited.

Communicator 41 may be connected to information terminal 30 to be able to perform wireless communication with information terminal 30 or may be connected to information terminal 30 to be able to perform wired communication with information terminal 30. For example, when communicator 41 is connected to information terminal 30 to be able to perform wired communication with information terminal 30, the wired communication is realized, for example, by a connector connected to a communication line or the like.

Information processor 42 is a processor which performs various types of information processing in determination device 40. For example, information processor 42 is realized, for example, by a microcomputer. Information processor 42 may also be realized by a processor. For example, the microcomputer, the processor, or the like of information processor 42 executes computer programs stored in storage 43 to realize the functions of information processor 42.

Information processor 42 includes acquirer 42a, estimator 42b, setter 42c, identifier 42d, determiner 42e, and outputter 42f.

Acquirer 42a is a processor which acquires the image from information terminal 30 via communicator 41. Specifically, acquirer 42a acquires, via communicator 41, the image (for example, moving images including a plurality of images) output (transmitted) from information terminal 30.

Estimator 42b is a processor which estimates (calculates), based on the image including, as the subject, the target person performing the specific action, a skeletal model of the target person in the image. Specifically, estimator 42b estimates, based on the imaged acquired by acquirer 42a, a skeletal model of the target person in the image. More specifically, estimator 42b estimates, based on moving images including a plurality of images, a skeletal model in each of the images included in the moving images.

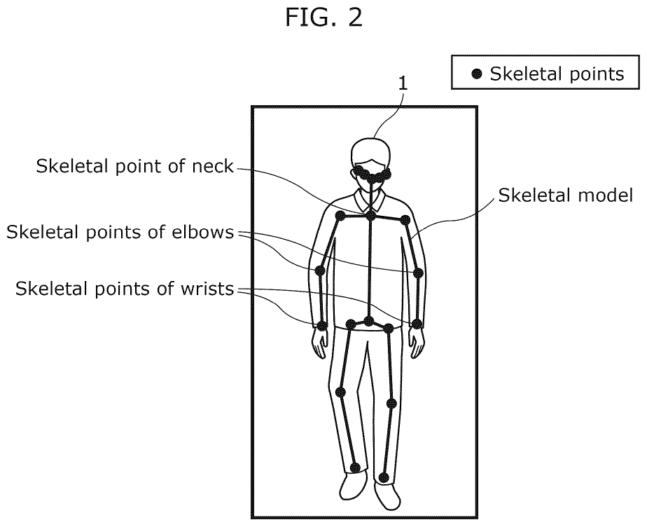

FIG. 2 is a diagram for illustrating a skeletal model of target person 1 which is estimated by estimator 42b in the embodiment. Specifically, FIG. 2 is a diagram which shows the skeletal model of target person 1 estimated by estimator 42b that is superimposed on target person 1, and schematically shows an image including target person 1 as the subject.

The skeletal model is a model which is generated by connecting, with links (lines), a plurality of skeletal points that are specific positions of joints and the like of target person 1 in the image. Specifically, the skeletal model is coordinate data of a plurality of skeletal points and the like. For example, estimator 42b performs image analysis and the like to estimate the positions (more specifically, the coordinates) of a plurality of skeletal points of target person 1 in the image which include the skeletal point of the neck, the skeletal points of the elbows, the skeletal points of the wrists, and the like and are previously determined. Furthermore, for example, estimator 42b connects, with lines, predetermined skeletal points such as the skeletal points of the elbows and the skeletal points of the wrists among the estimated skeletal points. In this way, estimator 42b estimates the skeletal model of target person 1.

For the estimation of the skeletal model, for example, an existing posture and a skeletal estimation algorithm may be used, or the skeletal model may be estimated by any method.

Estimator 42b may estimate a two-dimensional skeletal model of the target person or may estimate a three-dimensional skeletal model of the target person. In other words, estimator 42b may estimate two-dimensional coordinates of the skeletal points of the target person in the image or may estimate three-dimensional coordinates of the skeletal points of the target person. For example, estimator 42b estimates a two-dimensional skeletal model (that is, coordinates of the skeletal points in a two-dimensional orthogonal coordinate system) of the target person based on the image acquired by acquirer 42a, and estimates a three-dimensional skeletal model (that is, coordinates of the skeletal points in a three-dimensional orthogonal coordinate system) of the target person based on the estimated two-dimensional skeletal model using learned model 44 which is a learned machine learning model.

Learned model 44 is an identifier which is previously constructed by machine learning in which a two-dimensional skeletal model with known three-dimensional coordinate data of joints is used as learning data and the three-dimensional coordinate data is used as teacher data. Learned model 44 uses the two-dimensional skeletal model as an input to output three-dimensional coordinate data corresponding to the two-dimensional skeletal model, that is, the three-dimensional skeletal model. For example, learned model 44 is previously stored in storage 43.

As described above, estimator 42b may estimate the three-dimensional skeletal model of the target person in the image acquired by acquirer 42a.

Setter 42c is a processor which sets (calculates) a plurality of three-dimensional regions around the skeletal model based on positions of a plurality of skeletal points in the skeletal model estimated by estimator 42b.

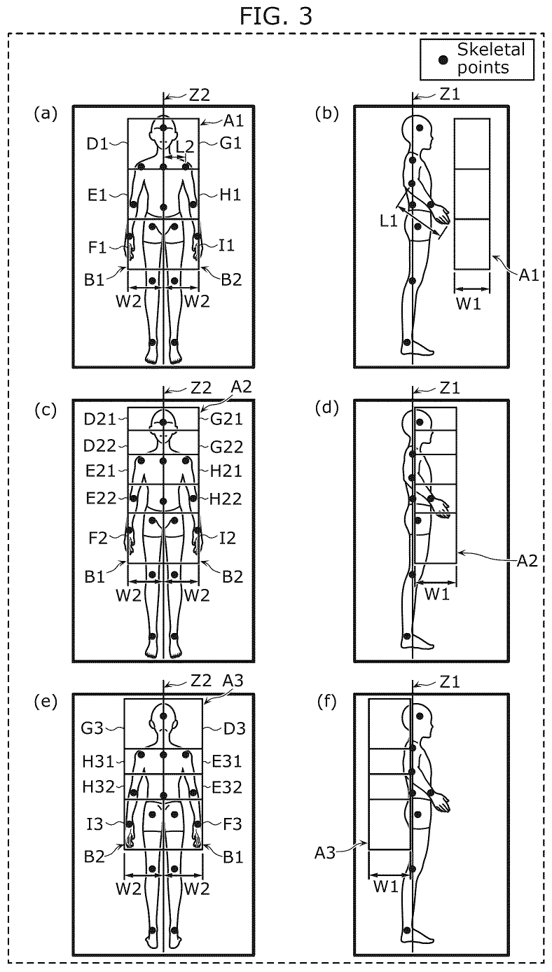

FIG. 3 is a diagram for illustrating three-dimensional regions which are set by setter 42c in the embodiment. Specifically, FIG. 3 is a diagram which shows the three-dimensional regions set by setter 42c and the skeletal points of the target person estimated by estimator 42b that are superimposed on each other, and schematically shows images including target person 1 as the subject. Parts (b), (d), and (f) in FIG. 3 are diagrams when an image of a side of the target person is captured, parts (a) and (c) in FIG. 3 are diagrams when an image of the front of the target person is captured, and part (e) in FIG. 3 is a diagram when an image of the back of the target person is captured. Parts (a) and (b) in FIG. 3 are diagrams schematically showing forward region A1 among the three-dimensional regions set by setter 42c. Parts (c) and (d) in FIG. 3 are diagrams schematically showing front surface region A2 among the three-dimensional regions set by setter 42c. Parts (e) and (f) in FIG. 3 are diagrams schematically showing back surface region A3 among the three-dimensional regions set by setter 42c.

As shown in parts (b), (d), and (f) in FIG. 3, for example, in a side view of the target person, setter 42c sets, as three-dimensional regions, back surface region A3 on the back surface side of the target person and front surface region A2 on the front surface side which are provided through first reference axis Z1 in a direction (also referred to as a longitudinal direction) that extends from the head of the target person to the legs of the target person and passes through a base point (first base point) and forward region A1 provided adjacent to the region on the front surface side on the front side of the target person.

The first base point may be determined previously and arbitrarily, and is not particularly limited. The number of first base points, that is, the number of skeletal points serving as first base points may be one or plural, and is not particularly limited. The first base points are, for example, the skeletal point of the neck and the skeletal point of the waist of the target person. In other words, for example, first reference axis Z1 is set based on the positions of the skeletal point of the neck and the skeletal point of the waist of the target person. Specifically, for example, in the side view of the target person, first reference axis Z1 is set to pass through the skeletal point of the neck and the skeletal point of the waist of the target person.

As shown in parts (a), (c), and (e) in FIG. 3, for example, in a front view of the target person, setter 42c sets back surface region A3, front surface region A2, and forward region A1 such that these regions include (for example, overlap left side region B2 and right side region B1) left side region B2 and right side region B1 which are provided adjacent to each other through second reference axis Z2 in the longitudinal direction passing through the base point (second base point). In other words, in the front view of the target person, setter 42c sets left side region B2 and right side region B1 provided adjacent to each other through second reference axis Z2 in the longitudinal direction passing through the base point (second base point) such that each of left side region B2 and right side region B1 includes back surface region A3, front surface region A2, and forward region A1.

The second base point may be determined previously and arbitrarily, and is not particularly limited. The number of second base points, that is, the number of skeletal points serving as second base points may be one or plural, and is not particularly limited. The second base points are, for example, the skeletal point of the neck and the skeletal points of the elbows of the target person. In other words, for example, second reference axis Z2 is set based on the positions of the skeletal point of the neck and the skeletal points of the elbows of the target person. Specifically, for example, in the front view of the target person, second reference axis Z2 is set to pass through the skeletal point of the neck and the intermediate of the skeletal point of the elbows of the target person.

As shown in parts (a) and (b) in FIG. 3, in this example, setter 42c sets three three-dimensional regions in each of left side region B2 and right side region B1 by dividing each of left side region B2 and right side region B1 in forward region A1 in a lateral direction orthogonal to the longitudinal direction.

As shown in parts (c) and (d) in FIG. 3, in this example, setter 42c sets five three-dimensional regions in each of left side region B2 and right side region B1 by dividing each of left side region B2 and right side region B1 in front surface region A2 in the lateral direction orthogonal to the longitudinal direction.

As shown in parts (e) and (f) in FIG. 3, in this example, setter 42c sets four three-dimensional regions in each of left side region B2 and right side region B1 by dividing each of left side region B2 and right side region B1 in back surface region A3 in the lateral direction orthogonal to the longitudinal direction.

As described above, for example, setter 42c uses at least one of a plurality of skeletal points in the skeletal model as the base point to set a plurality of three-dimensional regions D1, D21, D22, D3, E1, E21, E22, E31, E32, F1, F2, F3, G1, G21, G22, G3, H1, H21, H22, H31, H32, I1, I2, and I3 around the skeletal model.

One or more skeletal points serving as the first base points and one or more skeletal points serving as the second base points may be all the same or different, or part thereof may be the same, and the others may be different.

The positions, the sizes and the number of the three-dimensional regions set by setter 42c may be determined arbitrarily, and are not particularly limited.

For example, in the side view of the target person, setter 42c sets first distance L1 from the skeletal point of the elbow of the target person to a tip of a hand of the target person as width W1 of each of back surface region A3, front surface region A2, and forward region A1. For example, in the front view of the target person, setter 42c sets a distance twice second distance L2 from the skeletal point of the neck of the target person to the skeletal point of a shoulder of the target person as width W2 of each of left side region B2 and right side region B1.

The sizes and the shapes of the three-dimensional regions set by setter 42c may be the same as or different from each other.

Identifier 42d is a processor which identifies, among the three-dimensional regions set by setter 42c, a three-dimensional region (target three-dimensional region) where the skeletal point of the wrist of the target person is located in the specific action (that is, while the specific action is being performed). Specifically, identifier 42d identifies, based on the three-dimensional coordinate data (that is, the three-dimensional skeletal model) of the target person in the image, in which one of the three-dimensional regions the coordinates of the skeletal point of the wrist of the target person are located (that is, are included). For example, identifier 42d identifies, based on the three-dimensional skeletal model (that is, the three-dimensional coordinate data) of the target person in moving images which include, as the subject, the target person performing the specific action, one or more three-dimensional regions, among a plurality of three-dimensional regions, in which the skeletal point of the wrist of the target person is located while the specific action is being performed.

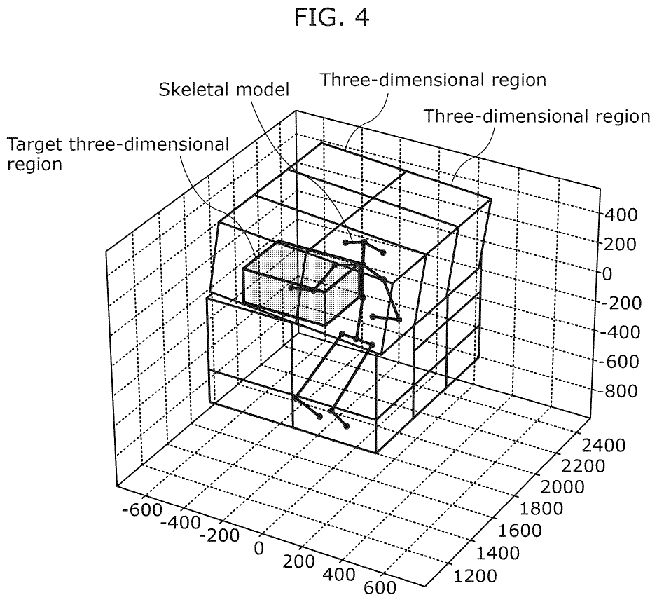

FIG. 4 is a diagram for illustrating the three-dimensional region which is identified by identifier 42d in the embodiment. In FIG. 4, the target three-dimensional region is hatched. The numerical values shown in FIG. 4 are numerical values illustrating coordinates corresponding to axes in the three-dimensional orthogonal coordinate system.

For example, estimator 42b estimates the skeletal model of the target person (that is, the coordinates of the skeletal points of the target person). Furthermore, setter 42c sets a plurality of three-dimensional regions. Furthermore, identifier 42d identifies, among the three-dimensional regions set by setter 42c, the target three-dimensional region in which the skeletal point of the wrist estimated by estimator 42b is located.

For example, identifier 42d identifies, among the three-dimensional regions set by setter 42c, the three-dimensional region through which the skeletal point of the wrist of the target person passes in the specific action.

For example, when the skeletal point of the wrist located in three-dimensional region F2 moves to three-dimensional region E22, and further reaches three-dimensional region E21, the three-dimensional region through which the skeletal point of the wrist of the target person passes is three-dimensional region E22.

Determiner 42e is a processor which determines (calculates), based on the three-dimensional region identified by identifier 42d, the degree to which the target person is capable of performing an activity of daily living. Specifically, determiner 42e determines (calculates), based on the three-dimensional region identified by identifier 42d, the degree to which the target person is capable of performing an activity of daily living and corresponds to the specific action. For example, determiner 42e determines, based on database 45 stored in storage 43, the degree to which the target person is capable of performing an activity of daily living.

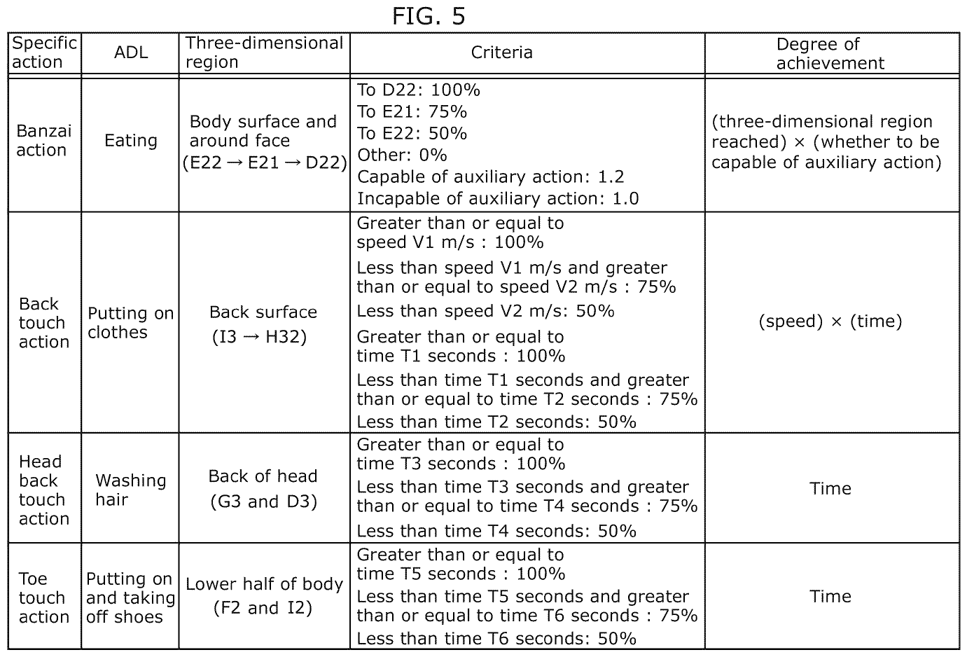

FIG. 5 is a diagram showing specific examples of criteria which are used by determiner 42e in the embodiment. More specifically, FIG. 5 is a diagram schematically showing database 45. An example shown in FIG. 5 indicates that as the degree of achievement which is the result of a determination when the target person performs the specific action is increased, the target person is capable of more correctly performing the activity of daily living corresponding to the specific action. For example, when the degree of achievement is 100%, it indicates that the target person is capable of correctly performing the activity of daily living corresponding to the specific action. For example, when the degree of achievement is 50%, it indicates that the target person is capable of performing the activity of daily living corresponding to the specific action to some extent, that is, the target person is not capable of performing the activity of daily living very well. For example, when the degree of achievement is 0%, it indicates that the target person is not capable of performing the activity of daily living corresponding to the specific action.

Database 45 is data which is stored in association with the specific action, the three-dimensional region (target three-dimensional region) in which the wrist is located in the specific action, the activity of daily living (ADL) corresponding to the specific action, and a method for calculating the degree corresponding to the specific action (the criteria and the degree of achievement).

The criteria are information indicating what determination is made by determiner 42e for each of a change in the position of the target three-dimensional region, a time period during which the position of the target three-dimensional region is not changed, a speed at which the position of the target three-dimensional region is changed, auxiliary action information which will be described later, and the like.

The degree of achievement is information indicating a method for calculating, by determiner 42e, the degree to which the target person is capable of performing the activity of daily living based on various types of information obtained by determinations made based on the criteria. In other words, the degree of achievement is, for example, information indicating a method for calculating the result of the determination made by determiner 42e.

For example, when the specific action is a “banzai action (action of raising both hands from a state where they are in lowered positions)”, if the skeletal point of the wrist of the target person moves from the body surface (the front of the torso) of the target person to an area around the face during the specific action (that is, while the target person is performing the specific action), determiner 42e determines that the target person is capable of performing 100% of an activity of “eating” as an activity of daily living. Specifically, for example, when the target person performs the “banzai action”, if the position of the skeletal point of the wrist moves from the initial position (for example, three-dimensional region F2), passes through three-dimensional region E22 and three-dimensional region E21 in this order, and reaches three-dimensional region D22 (“to D22: 100%” shown in FIG. 5), determiner 42e determines that the target person is capable of performing 100% of the activity of “eating”.

For example, when the target person performs the “banzai action”, if the position of the skeletal point of the wrist moves from the initial position, passes through three-dimensional region E22, and reaches three-dimensional region E21 but does not reach three-dimensional region D22 (“to E21: 75%” shown in FIG. 5), determiner 42e determines that the target person is capable of performing 75% of the activity of “eating”.

For example, when the target person performs the “banzai action”, if the position of the skeletal point of the wrist moves from the initial position, and reaches three-dimensional region E22 but does not reach three-dimensional region E21 (“to E22: 50%” shown in FIG. 5), determiner 42e determines that the target person is capable of performing 50% of the activity of “eating”.

As described above, when the target person is capable of performing the activity of daily living, determiner 42e determines to what degree the target person is capable of performing the activity of daily living.

Determiner 42e may determine whether the target person is capable of performing the activity of daily living. For example, when the target person is instructed to perform the “banzai action” but the position of the skeletal point of the wrist does not change from the initial position, that is, when the target three-dimensional region is not changed from three-dimensional region F2 (“other: 0%” shown in FIG. 5), determiner 42e determines that the target person is capable of performing 0% of the activity of “eating”.

Determiner 42e may determine the degree to which the target person is capable of performing the activity of daily living based on the auxiliary action information indicating whether the target person is capable of performing an auxiliary action related to the activity of daily living corresponding to the specific action and the three-dimensional region identified by identifier 42d.

Here, the auxiliary action is a so-called compensatory action, and is an action for helping the activity of daily living corresponding to the specific action. For example, the auxiliary action is different from the specific action corresponding to the activity of daily living.

For example, when the target person is capable of performing the “banzai action” which is an example of the specific action, since it is estimated that the target person is capable of performing an action of raising both hands, it can be considered that the target person is capable of performing the activity of “eating” which is an example of the activity of daily living corresponding to the “banzai action”, that is, an activity of moving a hand to bring food to the mouth. Here, for example, even if the target person is capable of only raising hands to the chest, that is, even if the target person is not capable of correctly performing the “banzai action”, when the target person is capable of performing an action of lowering the head, it can be considered that the target person is capable of performing the activity of “eating” more correctly than a case where the target person is not capable of performing the action of lowering the head. Hence, for example, when determiner 42e determines the degree of the activity of daily living, determiner 42e makes the determination with consideration given to not only the specific action but also whether the auxiliary action is capable of being performed. Determiner 42e determines, for example, based on the three-dimensional region identified by identifier 42d, to what degree the target person is capable of performing the specific action, assigns weights corresponding to whether the target person is capable of performing the auxiliary action, and thereby determines the degree to which the target person is capable of performing an activity of daily living.

For example, when the target person performs the “banzai action”, even if the position of the skeletal point of the wrist moves from the initial position, passes through three-dimensional region E22, and reaches three-dimensional region E21 but does not reach three-dimensional region D22, determiner 42e determines, when the target person is capable of performing the auxiliary action (“capable of auxiliary action: 1.2” shown in FIG. 5), that the target person is capable of performing 75%×1.2 (“(three-dimensional region reached)×(whether to be capable of auxiliary action)” shown in FIG. 5)=90% of the activity of “eating”. In other words, in this case, the result of the determination made by determiner 42e is 90%. On the other hand, for example, when the position of the skeletal point of the wrist moves from the initial position, passes through three-dimensional region E22, and reaches three-dimensional region E21 but does not reach three-dimensional region D22, and the target person is not capable of performing the auxiliary action (“incapable of auxiliary action: 1.0” shown in FIG. 5), determiner 42e determines that the target person is capable of performing 75%×1.0=75% of the activity of “eating”. In other words, in this case, the result of the determination made by determiner 42e is 75%.

The auxiliary action information may be previously stored in storage 43 or may be acquired from the user via receiver 34 or the like. Determination device 40 may determine, based on an image (for example, moving images) which includes, as the subject, the target person performing the auxiliary action, whether the target person is capable of performing the auxiliary action to acquire the result of the determination as the auxiliary action information. For example, estimator 42b estimates, based on the image which includes, as the subject, the target person performing the auxiliary action, the skeletal model of the target person in the image. Furthermore, for example, setter 42c sets a plurality of three-dimensional regions around the skeletal model based on positions of a plurality of skeletal points in the skeletal model estimated by estimator 42b. Furthermore, for example, identifier 42d identifies, among the three-dimensional regions, a three-dimensional region (auxiliary three-dimensional region) in which a specific skeletal point (for example, the skeletal point of the neck) of the skeletal points is located in the auxiliary action. Furthermore, for example, determiner 42e determines, based on the three-dimensional region identified by identifier 42d, whether the target person is capable of performing the auxiliary action. The specific skeletal point may be determined arbitrarily.

For example, determination device 40 uses the image used for the determination of the degree to which the target person is capable of performing the activity of daily living to determine whether the target person is capable of performing the auxiliary action. For example, furthermore, identifier 42d identifies, among the three-dimensional regions set by setter 42c, the auxiliary three-dimensional region in which one or more specific skeletal points of the skeletal points other than the skeletal point of the wrist are located in the specific action. Furthermore, for example, determiner 42e determines, based on the auxiliary three-dimensional region, whether the target person is capable of performing the auxiliary action. In other words, for example, determiner 42e determines, based on the target three-dimensional region and the auxiliary three-dimensional region, the degree to which the target person is capable of performing the activity of daily living. For example, when the skeletal point of the neck of the target person who is performing the “banzai action” is located in any one of three-dimensional regions E21, E22, H21, and H22, determiner 42e determines that the target person is capable of performing the auxiliary action, and when the skeletal point of the neck is located in a three-dimensional region other than those described above, determiner 42e determines that the target person is not capable of performing the auxiliary action. Information indicating that when the auxiliary three-dimensional region coincides with a certain specific three-dimensional region, the auxiliary action is determined to be capable of being performed may be previously stored in storage 43.

For example, the criteria may be the time period during which the position of the target three-dimensional region is not changed and/or the speed at which the position of the target three-dimensional region is changed. For example, the criteria may be combinations between the criteria described above and the assigned weights.

For example, when the specific action is a “back touch action (action of touching the back from a state where both hands are down)”, determiner 42e determines to what degree the target person is capable of performing “putting on clothes” based on the speed at which the skeletal point of the wrist of the target person moves on the back surface of the target person in the specific action and the time period during which the position thereof is held at the back. Specifically, when the target person performs the “back touch action”, determiner 42e determines to what degree the target person is capable of performing “putting on clothes” based on the speed at which the position of the skeletal point of the wrist passes through three-dimensional region I3 and the time period during which the position of the skeletal point of the wrist continues to be located in three-dimensional region H32 when the position of the skeletal point of the wrist moves from the initial position, passes through three-dimensional region I3, and reaches three-dimensional region H32.

The speed at which the position of the skeletal point of the wrist passes through three-dimensional region I3 is calculated, for example, based on the size of three-dimensional region I3 and a time elapsed after the position of the skeletal point of the wrist is located in three-dimensional region I3 until the position of the skeletal point of the wrist is located in three-dimensional region H32.

The time period during which the skeletal point of the wrist continues to be located in three-dimensional region H32 is, for example, a time elapsed after the skeletal point of the wrist is located in three-dimensional region I3 until the skeletal point of the wrist leaves three-dimensional region I3.

For example, when the speed at which the skeletal point of the wrist passes through three-dimensional region I3 is less than speed V1 m/s and greater than or equal to speed V2 m/s, and the time period during which the skeletal point of the wrist continues to be located in three-dimensional region H32 is less than time T2 seconds, determiner 42e determines 75%×50%=37.5% as the degree to which the target person is capable of performing the activity of “putting on clothes”.

As described above, for example, determiner 42e determines the degree to which the target person is capable of performing the activity of daily living based on the speed at which the skeletal point of the wrist passes through a first three-dimensional region among the three-dimensional regions.

For example, determiner 42e also determines the degree to which the target person is capable of performing the activity of daily living based on the time period during which the skeletal point of the wrist continues to be located in a second three-dimensional region among the three-dimensional regions.

The positions and the number of first three-dimensional regions may be arbitrarily determined according to the specific action. For example, determiner 42e may determine the degree to which the target person is capable of performing the activity of daily living based on speeds at which the skeletal point of the wrist passes through a plurality of three-dimensional regions (for example, the average of the speeds at which the skeletal point of the wrist passes through the three-dimensional regions or the lowest of the speeds at which the skeletal point of the wrist passes through the three-dimensional regions).

The position of the second three-dimensional region may be arbitrarily determined according to the specific action.

For example, when the specific action is a “head back touch action (action of touching the back of the head from a state where both hands are down)”, determiner 42e determines to what degree the target person is capable of performing “washing hair” based on the time period during which the skeletal points of the wrists of the target person continue to be located at the back of the head of the target person in the specific action. Specifically, when the target person performs the “head back touch action”, determiner 42e determines to what degree the target person is capable of performing “washing hair” based on the time period during which the skeletal points of the wrists continue to be located in three-dimensional regions G3 and D3. For example, when the time period during which the skeletal points of the wrists continue to be located in three-dimensional regions G3 and D3 is less than time T4 seconds, determiner 42e determines 50% as the degree to which the target person is capable of performing the activity of “washing hair”.

For example, when the specific action is a “toe touch action (action of touching toes from a state where both hands are down)”, determiner 42e determines to what degree the target person is capable of performing “putting on and taking off shoes” based on the time period during which the skeletal points of the wrists of the target person continue to be located at the lower half of the body of the target person in the specific action. Specifically, when the target person performs the “toe touch action”, determiner 42e determines to what degree the target person is capable of performing “putting on and taking off shoes” based on the time period during which the skeletal points of the wrists continue to be located in three-dimensional regions F2 and I2. For example, when the time period during which the skeletal points of the wrists continue to be located in three-dimensional regions F2 and I2 is greater than or equal to time T5 seconds, determiner 42e determines 100% as the degree to which the target person is capable of performing the activity of “putting on and taking off shoes”.

The specific actions, the activities of daily living, the three-dimensional regions, the criteria, and the degrees of achievement shown in FIG. 5 are only examples, are not particularly limited, and may be determined arbitrarily. For example, speeds V1 and V2 and times T1, T2, T3, T4, T5, and T6 shown in FIG. 5 may be determined arbitrarily. The value of a weight to be assigned may be changed depending on to what degree the target person is capable of performing the auxiliary action. For example, the weight in the case of “capable of auxiliary action” may be set to 1.0, and the weight in the case of “incapable of auxiliary action” may be set lower than 1.0.

The skeletal point of the wrist used for the determination made by determiner 42e may be the skeletal point of the right wrist, may be the skeletal point of the left wrist, or may be the skeletal points of both the left and right wrists.

Outputter 42f is a processor which outputs the result of the determination made by determiner 42e to information terminal 30 via communicator 41. Controller 32 acquires the output result of the determination, and causes presenter 35 to present the result of the determination. In this way, presenter 35 presents the result of the determination made by determiner 42e to the user.

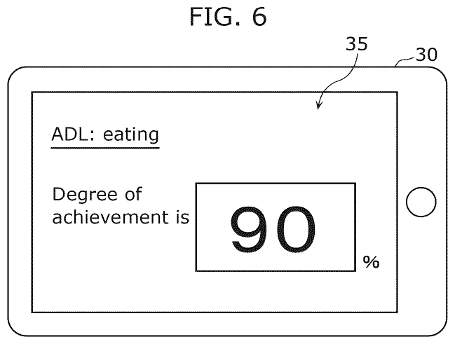

FIG. 6 is a diagram showing a specific example of the result of the determination which is presented by presenter 35 in the embodiment and is made by determiner 42e. The example shown in FIG. 6 is a specific example of an image in an example of the result of the determination which is presented by presenter 35 when determiner 42e determines 90% as the degree to which the target person is capable of performing the activity of “eating”.

As shown in FIG. 6, presenter 35 presents the results of the determination such as “ADL: eating” and “degree of achievement is 90%”, and thus the user can easily and accurately know the degree of the activity to which the target person is capable of performing the activity of daily living.

Outputter 42f may output the three-dimensional skeletal model in the moving images of the target person, a characteristic amount (for example, data of the physical function such as a joint movable range) used for the result of the determination of the state of activities of daily living, the result of the determination of the physical function of the target person, a rehabilitation training plan, or the like. Controller 32 may cause presenter 35 to present these pieces of information.

Storage 43 is a storage device which stores the image (image data) acquired by acquirer 42a, control programs to be executed by information processor 42, learned model 44, database 45, and information such as various types of threshold values described above. Storage 43 is realized, for example, by a semiconductor memory, an HDD, or the like.

### [Processing Procedure]

A processing procedure performed by determination system 10 will then be described.

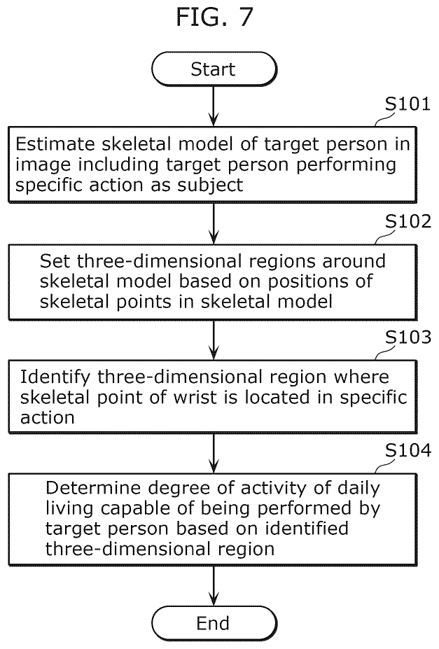

FIG. 7 is a flowchart showing the processing procedure which is performed by determination device 40 according to the embodiment.

Estimator 42b estimates, based on an image which includes, as a subject, the target person performing the specific action, a skeletal model of the target person in the image (S101). For example, estimator 42b estimates a two-dimensional skeletal model of the target person based on the image, and estimates, based on the estimated two-dimensional skeletal model, a three-dimensional skeletal model of the target person (that is, three-dimensional coordinate data indicating three-dimensional coordinates of a plurality of skeletal points) using learned model 44 which is a learned machine learning model.

Then, setter 42c sets a plurality of three-dimensional regions around the skeletal model based on positions of a plurality of skeletal points in the skeletal model estimated by estimator 42b (S102).

Then, identifier 42d identifies, among the three-dimensional regions set by setter 42c, a three-dimensional region in which a skeletal point of a wrist included in the skeletal points is located in the specific action (S103).

Then, determiner 42e determines, based on the three-dimensional region identified by identifier 42d, the degree to which the target person is capable of performing the activity of daily living (S104). For example, determiner 42e determines, based on the three-dimensional region identified by identifier 42d and database 45, the degree to which the target person is capable of performing the activity of daily living.

Determination device 40 may perform the processing in steps S101 to S104 as one-loop processing every time the target person performs each of a plurality of specific actions.

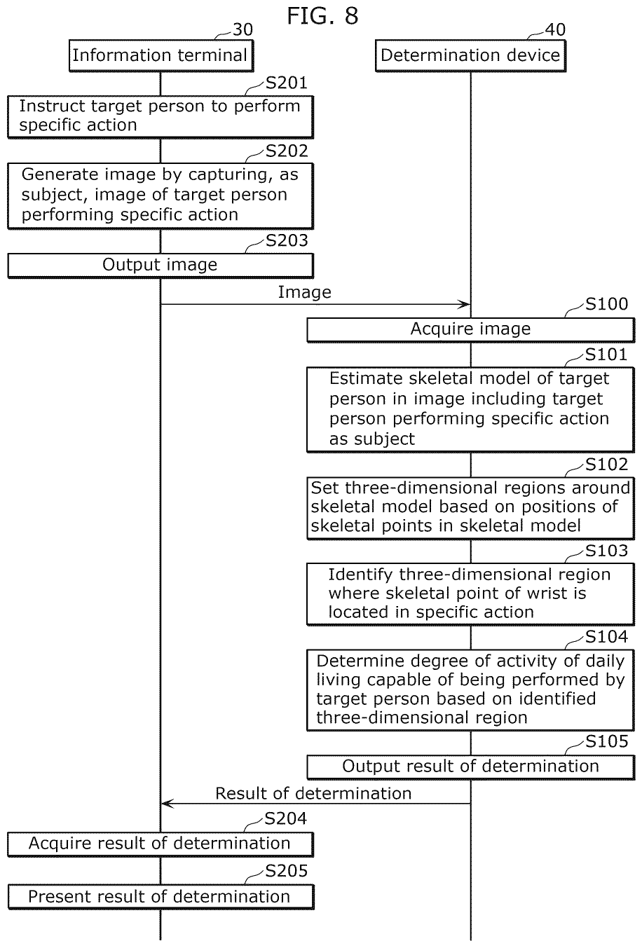

FIG. 8 is a flowchart showing a processing procedure which is performed by the determination system according to the embodiment.

Instructor 36 instructs the target person to perform the specific action (S201). Instructor 36 provides an instruction such as “take a banzai action”, for example, when receiver 34 receives, from the user, an instruction to perform processing for determining the degree to which the target person is capable of performing the activity of daily living (that is, an instruction to cause the target person to perform the specific action).

When receiver 34 receives the instruction, controller 32 may acquire an image captured by camera 20 to identify the target person in the acquired image. For the identification of the target person in the image, for example, a known image analysis technique is used.

Then, camera 20 captures, as the subject, an image of the target person performing the specific action to generate an image (more specifically, moving images) which includes, as the subject, the target person performing the specific action (S202).

Then, controller 32 outputs the image generated by camera 20 to determination device 40 via communicator 31 (S203). Here, controller 32 may anonymize the image and transmit it to determination device 40. In this way, the privacy data of the target person is protected.

Then, acquirer 42a acquires, via communicator 41, the image which is output by controller 32 via communicator 31 and is generated by camera 20 (S100).

Then, estimator 42b estimates the skeletal model of the target person in the image based on the image acquired by acquirer 42a, that is, the image which includes, as the subject, the target person performing the specific action (S101).

When acquirer 42a acquires moving images which include a plurality of images, estimator 42b may estimate, based on the acquired moving images, a skeletal model in each of the images included in the moving images.

Then, setter 42c sets a plurality of three-dimensional regions around the skeletal model based on positions of a plurality of skeletal points in the skeletal model estimated by estimator 42b (S102).

Then, identifier 42d identifies, among the three-dimensional regions set by setter 42c, a three-dimensional region in which the skeletal point of a wrist included in the skeletal points is located in the specific action (S103).

Then, determiner 42e determines, based on the three-dimensional region identified by identifier 42d, the degree to which the target person is capable of performing the activity of daily living (S104).

Then, outputter 42f outputs the result of the determination made by determiner 42e to information terminal 30 via communicator 41 (S105).

Then, controller 32 acquires, via communicator 31, the result of the determination output by outputter 42f via communicator 41 (S204).

Then, presenter 35 presents the result of the determination acquired by controller 32 (S205). Specifically, controller 32 causes presenter 35 to present the acquired result of the determination.

Information terminal 30 may perform the processing in steps S201 to S203 as one-loop processing every time the target person performs each of a plurality of specific actions. Alternatively, the processing in steps S201 and S202 may be performed for each of a plurality of specific actions, and after the target person completes all the specific actions, the processing in step S203 may be performed.

When the target person performs a plurality of specific actions, the result of the determination associated with each of the specific actions may be presented or only the result of the determination which is not satisfactory may be presented. These results of the determinations may be presented in order of inferior results.

Before information terminal 30 provides an instruction for the specific action, information terminal 30 may select, according to the physical function of the target person, the specific action which is caused to be performed by the target person. For example, before step S201, an instruction may be provided to the target person to perform an action of standing up from a sitting posture. Here, information terminal 30 may determine, based on the image of the target person captured by camera 20, whether the target person is capable of performing the action of standing up. Alternatively, the specific action which is caused to be performed by the target person may be selected based on an instruction received by receiver 34 and provided by the user.

In this way, the specific action can be selected according to the physical function of the target person, and thus it is possible to efficiently and accurately determine the state of activities of daily living of the target person.

### [Effects and Like]

As described above, the determination method according to the embodiment is a determination method performed by a computer, and the determination method includes: estimating, based on an image that includes, as a subject, a target person performing a specific action, a skeletal model of the target person in the image (S101); setting a plurality of three-dimensional regions around the skeletal model based on positions of a plurality of skeletal points in the skeletal model (S102); identifying, among the plurality of three-dimensional regions, a three-dimensional region where a skeletal point of a wrist included in the plurality of skeletal points is located in the specific action (S103); and determining, based on the three-dimensional region identified in the identifying, a degree to which the target person is capable of performing an activity of daily living (S104).

While an activity of daily living such as eating, putting on clothes, toileting, bathing or grooming is being performed, the wrist is easily located in a specific position corresponding to the activity of daily living relative to the body (for example, the torso) of the target person. Hence, in the determination method according to the embodiment, the skeletal model of the target person is estimated, the three-dimensional regions are set around the estimated skeletal model, and in which one of the set three-dimensional regions the skeletal point of the wrist is located is identified. In this way, it is possible to easily and accurately identify where the wrist is located relative to the body of the target person while the target person is performing the specific action. Hence, in the determination method according to an aspect of the present invention, it is possible to easily and accurately determine the state of activities of daily living of the target person.

For example, in the identifying, among the plurality of three-dimensional regions, a three-dimensional region through which the skeletal point of the wrist passes in the specific action is identified.

While the activity of daily living is being performed, the wrist easily passes through the specific position corresponding to the activity of daily living relative to the body (for example, the torso) of the target person. Hence, based on the three-dimensional region through which the skeletal point of the wrist passes in the specific action among the three-dimensional regions, it is possible to further accurately determine the state of activities of daily living of the target person.

For example, in the determining, the degree to which the target person is capable of performing the activity of daily living is determined based on a speed at which the skeletal point of the wrist passes through a first three-dimensional region among the plurality of three-dimensional regions.

For example, even when the target person is capable of performing the specific action, if the time it takes to complete the specific action after the start of the specific action is excessively long, it is difficult to say that the target person is capable of performing the activity of daily living corresponding to the specific action to the same degree as, for example, a healthy person. Hence, for example, in a specific action in which the skeletal point of the wrist passes through a specific three-dimensional region, based on the speed at which the skeletal point of the wrist passes through the specific three-dimensional region, the degree to which the target person is capable of performing the activity of daily living is determined. In this way, it is possible to further accurately determine the state of activities of daily living of the target person.

For example, in the determining, the degree to which the target person is capable of performing the activity of daily living is determined based on a time period during which the skeletal point of the wrist continues to be located in a second three-dimensional region among the plurality of three-dimensional regions.

For example, in an activity of daily living such as washing hair, it is necessary to keep the wrist in a specific position. Hence, for example, in a specific action in which the skeletal point of the wrist continues to be located in a specific three-dimensional region, based on the time period during which the skeletal point of the wrist continues to be located in the specific three-dimensional region, the degree to which the target person is capable of performing the activity of daily living is determined. In this way, it is possible to further accurately determine the state of activities of daily living of the target person.

For example, in the determining, the degree to which the target person is capable of performing the activity of daily living is determined based on auxiliary action information and the three-dimensional region identified in the identifying, the auxiliary action information indicating whether the target person is capable of performing an auxiliary action related to an activity of daily living corresponding to the specific action.

For example, in an activity of daily living such as eating, it is necessary to be able to move a hand to the mouth. Hence, when the target person is capable of performing the “banzai action” which is an example of the specific action, since it is estimated that the target person is capable of performing an action of raising both hands, it can be considered that the target person is capable of performing the activity of “eating” which is an example of the activity of daily living corresponding to the “banzai action”, that is, an activity of moving a hand to bring food to the mouth. Here, for example, even if the target person is capable of only raising hands to the chest, that is, even if the target person is not capable of correctly performing the “banzai action”, when the target person is capable of performing an action of lowering the head, it can be considered that the target person is capable of performing the activity of “eating” more correctly than a case where the target person is not capable of performing the action of lowering the head. Hence, for example, when determiner 42e determines the degree of the activity of daily living, determiner 42e makes the determination with consideration given to not only the specific action but also whether the auxiliary action is capable of being performed. In this way, it is possible to further accurately determine the state of activities of daily living of the target person.

For example, the determination method according to the embodiment further includes: outputting a result of determination made in the determining.

In this way, the user can easily know the state of activities of daily living of the target person.

Determination device 40 according to the embodiment includes: estimator 42b that estimates, based on an image which includes, as a subject, a target person performing a specific action, a skeletal model of the target person in the image; setter 42c that sets a plurality of three-dimensional regions around the skeletal model based on positions of a plurality of skeletal points in the skeletal model; identifier 42d that identifies, among the plurality of three-dimensional regions, a three-dimensional region where a skeletal point of a wrist included in the plurality of skeletal points is located in the specific action; and determiner 42e that determines, based on the three-dimensional region identified by identifier 42d, a degree to which the target person is capable of performing an activity of daily living.

Determination system 10 according to the embodiment includes; determination device 40; and information terminal 30, determination device 40 includes estimator 42b, setter 42c, identifier 42d, determiner 42e, a first communicator (communicator 41) that communicates with information terminal 30, acquirer 42a that acquires the image from information terminal 30 via the first communicator, and outputter 42f that outputs a result of the determination made by determiner 42e to information terminal 30 via the first communicator, and information terminal 30 includes a second communicator (communicator 31) that communicates with determination device 40, instructor 36 that instructs the target person to perform the specific action, camera 20 that generates the image by capturing an image of the target person performing the specific action, controller 32 that outputs the image generated to determination device 40 via the second communicator and acquires the result of the determination from determination device 40 via the second communicator, and presenter 35 that presents the result of the determination.

In this way, the same effects as in the determination method according to the embodiment described above are achieved.

### Other Embodiments

Although the embodiment has been described above, the present invention is not limited to the embodiment described above.

For example, although in the embodiment described above, the degree to which the target person is capable of performing the ADL is determined based on the position of the skeletal point of the wrist in the three-dimensional skeletal model, the present invention is not limited to this configuration. For example, the determination device may determine the degree to which the target person is capable of performing the ADL based on the position of the skeletal point of the wrist in the two-dimensional skeletal model. For example, although in the embodiment described above, determination device 40 determines the degree to which the target person is capable of performing the ADL based on the three-dimensional region in which the skeletal point of the wrist is located among a plurality of three-dimensional regions in the three-dimensional orthogonal coordinate system, determination device 40 may determine the degree to which the target person is capable of performing the ADL based on the region in which the skeletal point of the wrist is located among a plurality of regions in a two-dimensional orthogonal coordinate system.

For example, the instruction to cause the target person to perform the specific action may be provided by the user. In this case, the information terminal does not need to include the instructor.

Information terminal 30 may transmit, to determination device 40, an image (moving images) of the target person when the target person performs the specific action together with information indicating the specific action.

For example, information terminal 30 may transmit, to determination device 40, information indicating an instruction to cause the target person to determine the degree to which the target person is capable of performing the ADL based on an instruction from the user received by receiver 34. In this case, for example, determination device 40 may transmit, to information terminal 30, information indicating an instruction to cause the target person to perform the specific action. In this case, based on the received information, information terminal 30 may use instructor 36 to cause the target person to perform the specific action and use camera 20 to capture an image of the target person.

Determiner 42e may calculate, based on the skeletal model estimated by estimator 42b, a characteristic amount which indicates the characteristic of the movement of the target person in the specific action, and may determine the physical function which is the ability of the target person to perform a physical activity based on the calculated characteristic amount. For example, determiner 42e calculates, as the characteristic amount, based on the skeletal model estimated by estimator 42b, an angle (joint angle) formed by two links connected to a predetermined skeletal point of the target person. Alternatively, for example, determiner 42e calculates, as the characteristic amounts, a distance between the predetermined skeletal point and a terminal part in the specific action, the range of a variation in the position of the predetermined skeletal point in the specific action, and the like. For example, determiner 42e determines the physical function of the target person based on whether each of the calculated values is greater than or equal to a predetermined threshold value or whether each of the calculated values is in a predetermined range.

In this way, for example, it is possible to provide, based on the physical function such as muscle strength, a training plan necessary for maintaining or enhancing the physical function to the target person who has no problem with activities of daily living. The predetermined skeletal point, the predetermined threshold value, and the predetermined range may be determined arbitrarily. These pieces of information may be previously stored in storage 43.

Determiner 42e may further determine the degree to which the target person is capable of performing the activity of daily living based on the result of a determination as to whether an action accompanied by a movement of fingers of the target person (for example, an action of opening and closing a hand (clasping and unclasping a hand) or an action of opposing fingers (OK sign)) is capable of being performed. For example, when receiver 34 of information terminal 30 receives an instruction to determine whether an action accompanied by a movement of fingers is capable of being performed, controller 32 causes instructor 36 to instruct the target person to perform the action accompanied by the movement of fingers. When information terminal 30 acquires an image which is captured by camera 20 and includes, as a subject, the target person who performs the action accompanied by the movement of fingers, information terminal 30 transmits, to determination device 40, the instruction received by receiver 34 and the image captured by camera 20. Determiner 42e of determination device 40 determines whether the target person is capable of performing the action of opening and closing a hand by using another unillustrated learned model different from learned model 44. Determiner 42e may identify, with another learned model, the shape and the size of a space between the index finger and the thumb to determine whether a tip of the index finger is attached to a tip of the thumb in the image, and thereby determine whether an action of opposing fingers is capable of being performed. The other learned model may be previously stored in storage 43.

In this way, it is possible to determine whether the target person is capable of grasping an object, and thus it is possible to further accurately determine the degree to which the target person is capable of performing the activity of daily living.

Information about the physical function of the target person may be previously stored in storage 43 or the information may be received by receiver 34 from the user and acquired by acquirer 42a from information terminal 30.

Determiner 42e may produce a training plan for rehabilitation based on the result of the determination. Here, for example, determiner 42e may produce, in addition to the result of the determination, a training plan for rehabilitation based on the physical function of the target person.

For example, in the embodiment described above, processing performed by a specific processor may be performed by another processor. The order of a plurality of processing steps may be changed, and a plurality of processing steps may be performed in parallel with each other.

For example, in the embodiment described above, constituent elements of a processor such as information processor 42 may be realized by executing software programs suitable for the constituent elements. A program executor such as a CPU or a processor may read and execute software programs recorded in a recording medium such as a hard disk or a semiconductor memory to realize the constituent elements.

The constituent elements may be realized by hardware. The constituent elements may be circuits (or integrated circuits). These circuits may form one circuit as a whole or may be separate circuits. These circuits may be general-purpose circuits or dedicated circuits.

The overall or specific form of the present invention may be realized by a system, a device, a method, an integrated circuit, a computer program, or a computer-readable non-transitory recording medium such as a CD-ROM. The overall or specific form of the present invention may be realized by any combination of a system, a device, a method, an integrated circuit, a computer program, and a recording medium.

For example, the present invention may be realized as a determination method, may be realized as a program for causing a computer to perform a determination method, or may be realized as a computer-readable non-transitory recording medium in which the program as described above is recorded.

Although in the embodiment described above, the example is shown where determination system 10 includes information terminal 30 and determination device 40, the determination system according to the present invention may be realized as a single device such as an information terminal or may be realized by a plurality of devices. For example, the determination system may be realized as a client server system. When the determination system is realized by a plurality of devices, there is no limitation as to how the constituent elements included in the determination system described in the above embodiment are allocated to the devices.

Embodiments obtained by performing various types of variations conceived by a person skilled in the art on the embodiments and embodiments realized by arbitrarily combining the constituent elements and the functions in the embodiments without departing from the spirit of the present invention are also included in the present invention.

### REFERENCE SIGNS LIST

1 target person
10 determination system
20 camera
30 information terminal
31, 41 communicator
35 presenter
36 instructor
40 determination device
42b estimator
42c setter
42d identifier
42e determiner
42f outputter
D1, D21, D22, D3, E1, E21, E22, E31, E32, F1, F2, F3, G1, G21, G22, G3, H1, H21, H22, H31, H32, I1, I2, I3 three-dimensional region

1. A determination method performed by a computer, the determination method comprising:
estimating, based on an image that includes a target person as a subject, a skeletal model of the target person in the image, target person performing a specific action;
setting a plurality of three-dimensional regions around the skeletal model based on positions of a plurality of skeletal points in the skeletal model;
identifying, among the plurality of three-dimensional regions, a three-dimensional region where a skeletal point of a wrist included in the plurality of skeletal points is located in the specific action; and
determining, based on the three-dimensional region identified in the identifying, a degree to which the target person is capable of performing an activity of daily living.

2. The determination method according to claim 1,
wherein in the identifying, among the plurality of three-dimensional regions, a three-dimensional region through which the skeletal point of the wrist passes in the specific action is identified.

3. The determination method according to claim 2,
wherein in the determining, the degree to which the target person is capable of performing the activity of daily living is determined based on a speed at which the skeletal point of the wrist passes through a first three-dimensional region among the plurality of three-dimensional regions.

4. The determination method according to claim 1,
wherein in the determining, the degree to which the target person is capable of performing the activity of daily living is determined based on a time period during which the skeletal point of the wrist continues to be located in a second three-dimensional region among the plurality of three-dimensional regions.

5. The determination method according to claim 1,
wherein in the determining, the degree to which the target person is capable of performing the activity of daily living is determined based on auxiliary action information and the three-dimensional region identified in the identifying, the auxiliary action information indicating whether the target person is capable of performing an auxiliary action related to an activity of daily living corresponding to the specific action.

6. The determination method according to claim 1, further comprising:
outputting a result of determination made in the determining.

7. A determination device comprising:
an estimator that estimates, based on an image which includes, as a subject, a target person performing a specific action, a skeletal model of the target person in the image;
a setter that sets a plurality of three-dimensional regions around the skeletal model based on positions of a plurality of skeletal points in the skeletal model;
an identifier that identifies, among the plurality of three-dimensional regions, a three-dimensional region where a skeletal point of a wrist included in the plurality of skeletal points is located in the specific action; and
a determiner that determines, based on the three-dimensional region identified by the identifier, a degree to which the target person is capable of performing an activity of daily living.

8. A determination system comprising:
the determination device according to claim 7; and
an information terminal,
wherein the determination device further includes:
a first communicator that communicates with the information terminal;
an acquirer that acquires the image from the information terminal via the first communicator; and
an outputter that outputs a result of determination made by the determiner to the information terminal via the first communicator, and
the information terminal includes
a second communicator that communicates with the determination device,
an instructor that instructs the target person to perform the specific action,
a camera that generates the image by capturing an image of the target person performing the specific action,
a controller that outputs the image generated to the determination device via the second communicator, and acquires the result of the determination from the determination device via the second communicator, and
a presenter that presents the result of the determination.
